(19) United States
(12) Patent Application Publication
(10) Pub. No.: US 2006/0204398 A1
(43) Pub. Date: Sep. 14, 2006

(54) Process and apparatus for reducing likelihood of transmission of infection between patients by an X-ray cassette

(76) Inventor: Mark P. Bales, Sand Springs, OK (US)

Correspondence Address: GABLE & GOTWALS, 100 WEST FIFTH STREET, 10TH FLOOR, TULSA, OK, 74103, US

(21) Appl. No.: 11/074,919

(22) Filed: Mar. 8, 2005

(51) Int. Cl. A61L 2/18; A61L 2/00; A61L 9/00

(52) U.S. Cl. 422/28

(57) ABSTRACT

To reduce the likelihood of transmission of infection from a patient, the exposed surface of an X-ray cassette is disinfected, the cassette is covered with a fluid repelling, radio lucent wrap and X-rays of the patient are taken while the cassette is still covered by the wrap. The cassette may then be removed from the wrap and re-covered with another fluid repelling, radio lucent wrap or, alternatively, left in the wrap and the wrap disinfected for subsequent use.

### BACKGROUND OF THE INVENTION

This invention relates generally to X-ray cassettes and more particularly concerns the transfer of infection from one patient to another by an X-ray cassette.

The taking of X-rays, especially in trauma situations, frequently involves contact between the X-ray cassette and the patient's body fluids and tissue. Each cassette is likely used for many patients, so the cassettes can easily become carriers of infections from patient to patient.

The present response to this problem is patient based. That is, corrective efforts are directed toward protecting the patient from the cassette. In a common course of X-ray practice, for example, a bleeding patient may be in isolation or in an emergency room. An X-ray technologist is called upon to X-ray the injury. The technologist takes the bare cassette, a plastic bag, disinfectant, a towel and a portable X-ray machine from the X-ray department to the patient. The technologist determines the isolation precaution level for the patient and puts on the necessary precautionary clothing such as gown, gloves and mask for the situation. If body fluids are a concern, it is expected that the cassette will be put in the plastic bag. Otherwise, it is not expected that the cassette will be inserted into the bag. With the patient positioned on the X-ray machine, either the bare cassette or the bagged cassette is placed in contact with the traumatized area of the patients body. If the bag is used, after taking the X-ray, the cassette is removed from the bag and the bag is discarded. The cassette should be cleaned by spraying its surface with the disinfectant and wiping it with the towel. The protective clothing is left in the patient area. The X-ray machine should be wiped down with disinfectant. The technologist's hands should be sanitized. The technologist returns to the X-ray department with the cassette and machine. The process will be repeated with the same cassette for a subsequent patient.

This process is fraught with problems. Whether by inadvertence, error in judgment or carelessness, protocols that “should” be followed may not be followed. It is impossible to know whether technologists who have previously used the cassette have followed all sanitizing protocols. It is impossible to be sure whether a cassette has ever been used without a bag and come into contact with a patient. It is impossible to be sure whether a cassette has been disinfected after every previous use. Even if the cassette has been disinfected, the best disinfecting effort only sanitizes the surface of the cassette. The cassette has nooks and crannies at the seams of its walls and surface components and openings into its working mechanisms, all of which can be exposed to a patient's blood and perspiration and cannot be reached by the disinfectant.

It is, therefore, an object of this invention to provide a method and apparatus for use in taking X-rays which reduce the likelihood of patient-cassette contact. Another object of this invention is to provide a method and apparatus for use in taking X-rays which reduce the likelihood that a cassette will become a carrier of infection. A further object of this invention is to provide a method and apparatus for use in taking X-rays which reduce the likelihood that a technologist will not follow appropriate sanitizing protocol. Yet another object of this invention is to provide a method and apparatus for use in taking X-rays which affirmatively indicate that a subsequent patient will not be exposed to body fluids from a previous patient. It is also an object of this invention to provide a method and apparatus for use in taking X-rays which reduce the likelihood that a patient's body fluids will come into contact with an X-ray cassette. Still another object of this invention is to provide a method and apparatus for use in taking X-rays which reduce the likelihood that a patient's body fluids will penetrate into nooks, crannies and openings in an X-ray cassette. An additional object of this invention is to provide a method and apparatus for use in taking X-rays which facilitate the exercise of suitable sanitizing protocols respecting X-ray cassettes.

### SUMMARY OF THE INVENTION

In accordance with the invention, a process and apparatus are provided for reducing the likelihood of transmission of infection from a patient.

In the process, the exposed surface of an X-ray cassette is disinfected. The disinfected cassette is covered with a fluid repelling, radio lucent wrap. X-rays of the patient are taken using the covered, disinfected cassette while the cassette is still covered by the wrap. The disinfected cassette may be removed from the wrap after taking the X-rays of the patient and after contact between the patient and the cassette is terminated. If removed, the disinfected cassette may be re-covered with another fluid repelling, radio lucent wrap. Alternatively, the cassette may be left in the wrap and the wrap disinfected for subsequent use.

Disinfecting may be accomplished by spraying disinfectant on the exposed surface of the X-ray cassette and drying the sprayed surface of the cassette. Covering may be accomplished by inserting the disinfected cassette into a bag formed from the fluid repelling, radio lucent wrap and folding the open mouth of the bag over itself or sealing the open mouth of the bag with adhesive to reduce a likelihood of fluids contacting the inserted cassette or by encasing the disinfected cassette in a shrink-wrapped package formed from the fluid repelling, radio lucent wrap. Re-covering, if applicable, may be accomplished by any of the alternatives usable for covering.

The apparatus is an X-ray cassette having a disinfected outer surface and a cover formed with a fluid repelling, radio lucent wrap. The cover may be a bag formed from the fluid repelling, radio lucent wrap having its open mouth folded over itself or sealed with adhesive or may be a shrink-wrapped package formed from the fluid repelling, radio lucent wrap.

### BRIEF DESCRIPTION OF THE DRAWINGS

Other objects and advantages of the invention will become apparent upon reading the following detailed description and upon reference to the drawings in which:

FIG. 1 is plan view of the front face of a typical known X-ray cassette;

FIG. 2 is a plan view of the back face of the cassette of FIG. 1;

FIG. 3 is an elevation view of the top end of the cassette of FIG. 1;

FIG. 4 is a block diagram illustrating the process for reducing the likelihood of transmission of infection from a patient;

FIG. 5 is a perspective view of a fold-over bag embodiment of the apparatus for reducing the likelihood of transmission of infection from a patient;

FIG. 6 is a perspective view of a sealed bag embodiment of the apparatus for reducing the likelihood of transmission of infection from a patient; and

FIG. 7 is a perspective view of a shrink-wrap embodiment of the apparatus for reducing the likelihood of transmission of infection from a patient

While the invention will be described in connection with preferred processes and structural embodiments thereof, it will be understood that it is not intended to limit the invention to those processes or embodiments or to the details of the steps of the processes or construction or arrangement of parts illustrated in the accompanying drawings.

### DETAILED DESCRIPTION

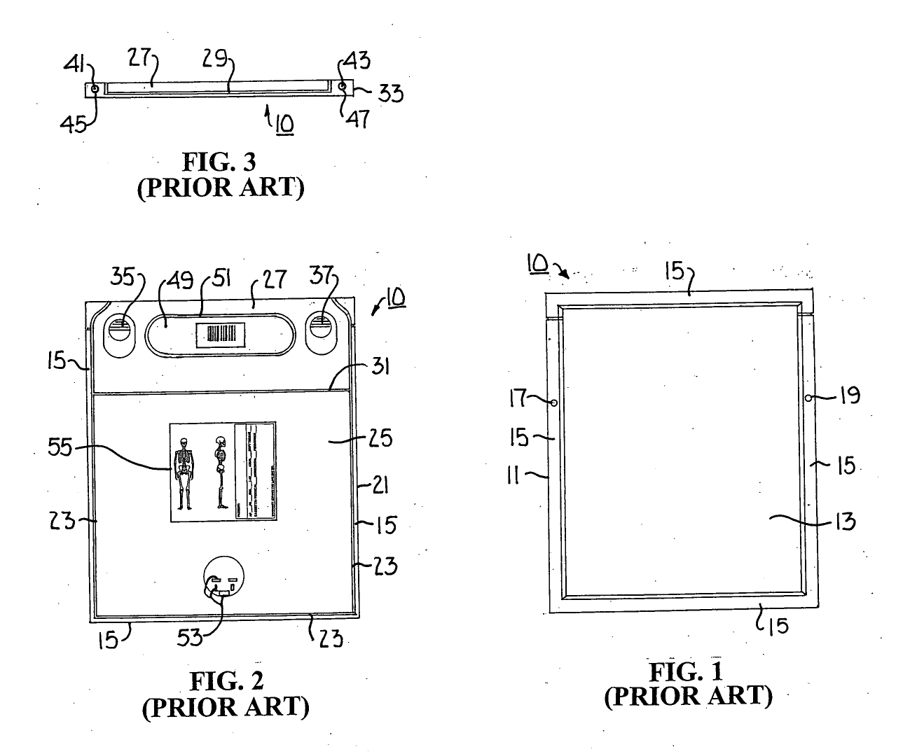

Turning first to FIGS. 1-3, the external structure of a typical known X-ray cassette 10 is illustrated. The front face 11 of the cassette 10 is the X-ray intensifying screen 13 surrounded by the outer frame 15. The frame 15 overlaps the periphery of the screen 13. Holes 17 and 19 are provided on opposite sides of the frame 15 for registering the position of the cassette 10 in other equipment (not shown). The back face 21 of the cassette 10 has a peripheral seam 23 between the cassette frame 15 and back panel 25. A cover 27 has a seam 29 along one edge 31 hinged to the back panel 25 and extending along the frame 15 and across the top end 33 of the cassette 10 where the X-ray film is inserted. Sliding latches 35 and 37 in the cover 27 operate pins 41 and 43 in apertures 45 and 47 of the frame 10 to lock and unlock the cover 27. The cover 27 also has a bar code viewing window 49 which overlaps an opening 51 in the cover 27. Other slots 53 may also be provided, as shown in the back panel 25, for registering the position of the cassette 10 in relation to other equipment (not shown). A label 55 is adhesively applied to the back panel 25 for temporarily recording data related to the patient being X-rayed.

Not all cassettes are exactly like the one described. However, all of them have nooks, crannies and crevices similar to those associated with the overlaps, holes, seams, covers, hinges, latches, windows, slots and labels of this typical cassette. All of these nooks, crannies and crevices are havens in which body fluids and the bacteria and infections they carry are shielded from disinfectant.

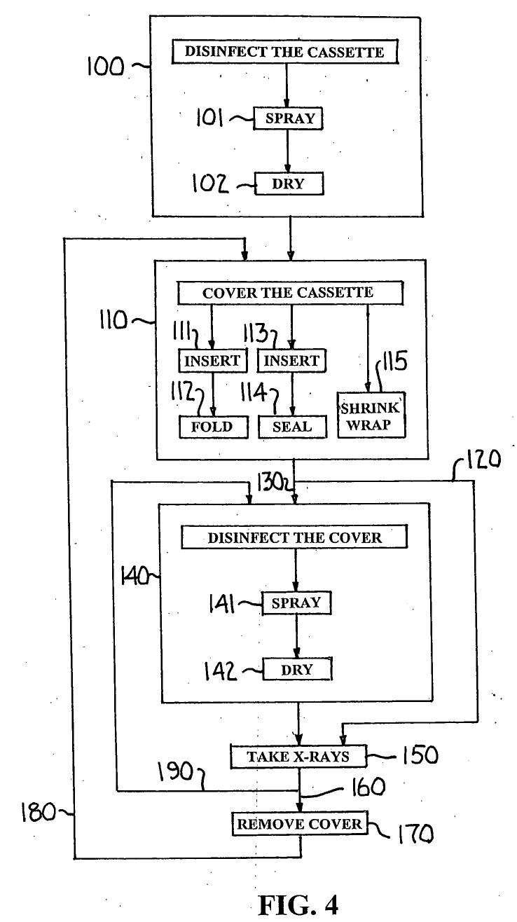

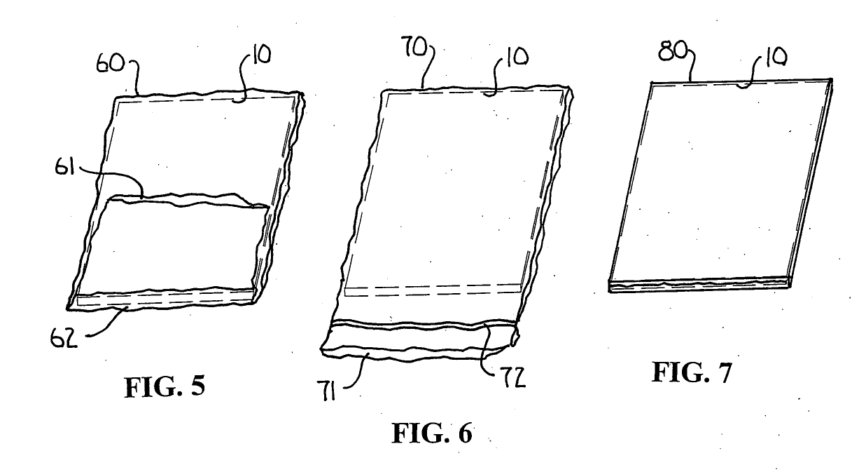

In order to reduce the likelihood that a cassette 10 being used to X-ray a patient remains contaminated from previous use with another patient, the present process, illustrated in FIG. 4, approaches disinfecting from the standpoint of protecting the cassette 10 rather than the patient. The first step is to disinfect 100 the exposed surface of the cassette 10, whether new or used, perhaps by spraying 101 the cassette with disinfectant and drying 102 the cassette. The next step is to cover 110 the disinfected cassette 10 with a fluid repelling, radio lucent wrap. This can be accomplished in one of several ways. One way, illustrated in FIG. 5, is to insert 111 the disinfected cassette 10 into a bag 60 formed from the fluid repelling, radio lucent wrap and then to fold 112 the open mouth 61 of the bag 60 over itself 62 to reduce likelihood of fluid contacting the inserted cassette 10. A second way, illustrated in FIG. 6, is to insert 113 the disinfected cassette 10 into a bag 70 formed from the fluid repelling, radio lucent wrap and then to seal 114 the open mouth 71 of the bag 70, perhaps with adhesive or in zip-lock fashion 72, to reduce a likelihood of fluids contacting the inserted cassette 10. A third way, illustrated in FIG. 7, is to encase 115 the disinfected cassette 10 in a shrink-wrapped package 80 formed from the fluid repelling, radio lucent wrap. The covered, disinfected cassette 10 may, in one application 120 of the process, be used to take X-rays 150 of the patient while the cassette 10 is still covered by the wrap, resulting in a reduced likelihood that the cassette 10 become contaminated.

In an alternative application 130 of the process, after the disinfected cassette 10 is covered 110, the additional precautionary step 140 may be taken to disinfect the fluid repelling, radio lucent cover 60, 70 or 80. Since the cover 60, 70 or 80 does not have the inaccessible nooks, crannies and crevices of the cassette, disinfecting the cover 60, 70 or 80 is more effective than disinfecting the cassette 10 directly. This is especially effective if the cover is a shrink wrap 80. Disinfecting 140 may, as in the case of disinfecting the cassette 10 directly, be accomplished by spraying 141 the cover 60, 70 or 80 with disinfectant and drying 142 the cover 60, 70 or 80. After taking this precautionary step, the cassette 10 with the disinfected cover 60, 70 or 80 may, in this alternative application 130, be used to take X-rays 150 of the patient.

Whether or not the cover 60, 70 or 80 has been disinfected before taking X-rays 150, after X-rays are taken 150 and contact between the patient and the cassette 10 is terminated, there are two alternatives 160 and 190 for the remainder of the process. In one alternative 160, after taking X-rays 150, the cover 60, 70 or 80 may be removed 170 from the cassette 10. The cassette 10 can then be re-covered by returning 180 to the cover the cassette step 110 of the procedure and proceeding with the same options available from the this step. In the other alternative 190, after taking X-rays 150, the cover 60, 70 or 80 can be re-disinfected by returning to the disinfect the cover step 140 of the procedure and proceeding with the same options available from the this step.

Thus, it is apparent that there has been provided, in accordance with the invention, a process and apparatus for reducing the likelihood of transmission of infection from a patient that fully satisfies the objects, aims and advantages set forth above. While the invention has been described in conjunction with specific processes and structural embodiments, it is evident that many alternatives, modifications and variations will be apparent to those skilled in the art and in light of the foregoing description. Accordingly, it is intended to embrace all such alternatives, modifications and variations as fall within the spirit of the appended claims.

What is claimed is:

1. A process for reducing the likelihood of transmission of infection from a patient comprising the steps of:
disinfecting the exposed surface of an X-ray cassette;
covering the disinfected cassette with a fluid repelling, radio lucent wrap; and
taking X-rays of the patient using the covered, disinfected cassette while the cassette is still covered by the wrap.

2. A process according to claim 1, said step of disinfecting comprising the sub-steps of:
spraying disinfectant on the exposed surface of the X-ray cassette; and
drying the sprayed surface of the cassette.

3. A process according to claim 1, said step of covering comprising the sub-steps of:
inserting the disinfected cassette into a bag formed from the fluid repelling, radio lucent wrap; and
folding an open mouth of the bag over itself to reduce a likelihood of fluids contacting the inserted cassette.

4. A process according to claim 1, said step of covering comprising the sub-steps of: wrap; and
inserting the disinfected cassette into a bag formed from the fluid repelling, radio lucent
sealing an open mouth of the bag to reduce a likelihood of fluids contacting the inserted cassette.

5. A process according to claim 1, said step of covering comprising encasing the disinfected cassette in a shrink-wrapped package formed from the fluid repelling, radio lucent wrap.

6. A process according to claim 1 further comprising the step of removing the cassette from the wrap after taking the X-rays of the patient and contact between the patient and the cassette is terminated.

7. A process according to claim 6 further comprising the step of re-covering the removed cassette with another fluid repelling, radio lucent wrap.

8. A process according to claim 7, said step of re-covering comprising the sub-steps of:
inserting the disinfected cassette into a bag formed from the fluid repelling, radio lucent wrap; and
folding an open mouth of the bag over itself to reduce a likelihood of fluids contacting the inserted cassette.

9. A process according to claim 7, said step of re-covering comprising the sub-steps of:
inserting the disinfected cassette into a bag formed from the fluid repelling, radio lucent wrap; and
sealing an open mouth of the bag to reduce a likelihood of fluids contacting the inserted cassette.

10. A process according to claim 7, said step of re-covering comprising encasing the disinfected cassette in a shrink-wrapped package formed from the fluid repelling, radio lucent wrap.

11. A process according to claim 1 further comprising the step of disinfecting the fluid repelling, radio lucent wrap with the cassette still covered by the wrap after taking X-rays of the patient.

12. A process for reducing the likelihood of transmission of infection from a patient comprising the steps of:
covering an X-ray cassette with a fluid repelling, radio lucent wrap;
disinfecting the fluid repelling, radio lucent wrap with the cassette still covered by the wrap; and
taking X-rays of the patient while the cassette is still covered by the disinfected wrap.

13. A process according to claim 12, said step of disinfecting comprising the sub-steps of:
spraying disinfectant on the exposed surface of the wrap; and
drying the sprayed surface of the wrap.

14. A process according to claim 12, said step of covering comprising the sub-steps of:
inserting the cassette into a bag formed from the fluid repelling, radio lucent wrap; and
folding an open mouth of the bag over itself to reduce a likelihood of fluids contacting the inserted cassette.

15. A process according to claim 12, said step of covering comprising the sub-steps of:
inserting the cassette into a bag formed from the fluid repelling, radio lucent wrap; and
sealing an open mouth of the bag to reduce a likelihood of fluids contacting the inserted cassette.

16. A process according to claim 12, said step of covering comprising encasing the cassette in a shrink-wrapped package formed from the fluid repelling, radio lucent wrap.

17. An apparatus for reducing the likelihood of transmission of infection from a patient comprising an X-ray cassette having an outer surface thereof disinfected and covered by a fluid repelling, radio lucent wrap.

18. An apparatus according to claim 17, said wrap comprising a bag, an open mouth of said bag being folded over itself.

19. An apparatus according to claim 17, said wrap comprising a bag, an open mouth of said bag being sealed.

20. An apparatus according to claim 17, said wrap comprising a shrink-wrapped package.
